(19) United States
(12) Patent Application Publication
(10) Pub. No.: US 2025/0325178 A1
(43) Pub. Date: Oct. 23, 2025

(54) PUPILLOMETER WITH INDEXED LIGHT REFLEXT OUTPUT IMMUNE TO MEDICATION-INDUCED PUPILLARY CONDITION

(71) Applicant: Neuroptics, Inc., Irvine, CA (US)

(72) Inventors: Kamran Siminou, Newport Coast, CA (US); Jeffrey Oliver, Sugar Land, TX (US); Claudio Privitera, Albany, CA (US)

(21) Appl. No.: 19/185,561

(22) Filed: Apr. 22, 2025

Related U.S. Application Data
(60) Provisional application No. 63/636,910, filed on Apr. 22, 2024.

(51) Int. Cl. A61B 3/11; A61B 3/14; G16H 30/00

(52) U.S. Cl. CPC A61B 3/112; A61B 3/14; G16H 30/00; A61B 2576/02

(57) ABSTRACT

A pupillometer comprising a camera, a light source, a display, and a microprocessor configured with an algorithm to generate an indexed output representing pupillary light reflex reactivity. The indexed output is substantially invariant to changes in baseline pupil size caused by pharmacologic agents such as opioids or by ambient lighting variations, thereby providing a robust and reliable measure of neurologic function across a wide range of clinical conditions.

### CROSS REFERENCE TO RELATED APPLICATIONS

This application claims priority from U.S. Provisional Patent Application Ser. No. 63/636,910, filed Apr. 22, 2024, the entirety of which is incorporated herein by reference.

### FIELD OF THE INVENTION

The present invention relates generally to diagnostic medical devices and methods for neurological assessment, and more particularly to pupillometers that generate a medication-resistant indexed output for evaluating pupillary light reflex reactivity.

### BACKGROUND OF THE INVENTION

The pupillary light reflex (PLR) is a critical clinical indicator of neurological function, often used in the assessment of midbrain integrity, brainstem responsiveness, and cranial nerve III functionality. Accurate and reliable measurement of the PLR is essential in the context of traumatic brain injury (including concussion), opioid intoxication, cardiac arrest, and other conditions affecting neurologic status.

Traditional assessment methods rely on visual inspection of pupil reactivity using penlights or flashlights, which are inherently subjective and prone to inter-observer variability. Quantitative pupillometry—particularly infrared pupillometry—has emerged as a method to improve precision and repeatability of PLR assessment.

However, current pupillometer systems exhibit significant variability when interpreting responses in patients with altered baseline pupil diameters. Such alterations may result from ambient light exposure, consensual light stimulation of the contralateral eye, or pharmacologic agents such as opioids, which constrict the pupil without necessarily indicating neurologic compromise. In these scenarios, devices that fail to account for baseline pupil size may produce misleading results.

Therefore, there is a need for a pupillometer capable of producing an indexed output that accurately reflects the quality of the light reflex and is robust to changes in baseline pupil diameter caused by pharmacological or environmental factors.

### SUMMARY OF THE DISCLOSURE

The present invention provides systems, methods, and devices for quantitative pupillometry with a medication-resistant indexed output. In one embodiment, the system includes a camera, a light source, a display, and a microprocessor executing an algorithm that evaluates pupil response to light stimulation and generates an indexed output indicative of pupillary reactivity, wherein the indexed output is not affected by a change in pupil size caused by administration of an opioid medication to the subject prior to stimulation with light from the light source.

The algorithm compensates for pupil size variation by applying normalization or correction techniques, enabling the device to generate consistent outputs even when baseline pupil diameter is altered by external light sources or opioid administration.

The invention enables accurate neurological assessment in clinical settings such as critical care units, emergency departments, and operating rooms, improving diagnostic confidence and minimizing false indications of neurologic impairment in patients with pharmacologically or environmentally induced miosis.

### BRIEF DESCRIPTION OF THE DRAWINGS

FIG. 1 illustrates indexed outputs (Index N and Index I) across variable pupil diameters as altered by remifentanil and ambient light conditions.

FIG. 2 shows time-series plots of Index N and Index I during remifentanil administration and subsequent recovery.

FIG. 3 demonstrates the relationship between indexed outputs and pupil diameter under two different pupil-constricting conditions: opioid administration and ambient light stimulation.

### DETAILED DESCRIPTION

In the following detailed description, numerous specific details are set forth by way of examples in order to provide a thorough understanding of the relevant teachings. However, it should be apparent to those skilled in the art that the present teachings may be practiced without such details. In other instances, well known methods, procedures, components, and circuitry have been described at a relatively high-level, without detail, in order to avoid unnecessarily obscuring aspects of the present teachings.

Additionally, various forms and embodiments of the invention are illustrated in the figures. It will be appreciated that the combination and arrangement of some or all features of any of the embodiments with other embodiments is specifically contemplated herein. Accordingly, this detailed disclosure expressly includes the specific embodiments illustrated herein, combinations and sub-combinations of features of the illustrated embodiments, and variations of the illustrated embodiments.

In one embodiment, a pupillometer comprises a high-resolution infrared camera, a calibrated light-emitting diode (LED) light source, a display screen, and an onboard microprocessor. The microprocessor is configured with software algorithms that analyze image frames captured before, during, and after the light stimulus. The light source emits a stimulus of controlled intensity and duration toward the subject's eye, and the camera captures the dynamic pupil response. The microprocessor extracts features including baseline pupil diameter, minimum constriction diameter, latency, constriction velocity, and dilation velocity, among other response features.

An indexed output is computed using a proprietary algorithm. In one embodiment, the algorithm normalizes pupil response relative to expected pupil response values under unstimulated conditions or derived from prior calibration data or machine learning models trained on a broad population dataset. Thus, the algorithm can include a machine learning model trained to distinguish between physiological and pharmacological causes of pupil constriction.

This indexed output is invariant to baseline pupil diameter, particularly in cases where pupil size is reduced due to opioid administration or light stimulation. This is possible because the algorithm applies a correction factor to compensate for opioid-induced miosis or consensual light stimulation. The indexed output can be scaled between 0 and 5, with scores below 3 indicating abnormal or reduced pupillary light reflex whereas scores 3 and above indicate a normal pupillary light reflex. Examples of opioid medications that can induce miosis include propofol. Other examples include, without limitation, Hydrocodone, Oxycodone, Morphine, Fentanyl, Codeine, M ethadone, Tramadol, Buprenorphine, Hydromorphone, Oxymorphone.

### EXAMPLE

In a comparative study, ten healthy volunteer subjects were evaluated using two commercially available pupillometers, designated as pupilometer N and pupilometer I. Pupilometer N provided an output referred to as Index N, and pupilometer I provided an output called Index I. Index N and Index I were calculated differently, with Index I being affected by administration of opiods, such as remifentanil, whereas Index N is not affected by administration of opiods, such as remifentanil. As shown below, the results estimate an approximate 0.8-unit decrease in Index I with each 1-mm decrease below 3.5 mm pupil size, but no significant change in Index N across a range of pupil sizes.

In the first five subjects, remifentanil was administered to induce pharmacologic miosis (constriction of the pupil caused by the administration of medication); in the remaining five subjects, pupil size was altered by varying ambient light directed toward the contralateral eye. Measurements were recorded every 2.5 minutes during drug infusion and across a 25-minute recovery period.

Measurements were taken sequentially with each pupilometer in 10 volunteer subjects. Five subjects received remifentanil, with indexed outputs N and I (“Index N and Index I” respectively) obtained from each instrument every 2.5 minutes during 10-minute infusion of remifentanil and 25-minute recovery. Prior studies have shown that the administration of remifentanil constricts the pupil to diameters that restrict the movement of the iris.

Another five subjects had measurements taken by each pupilometer as varying levels of light intensity were applied to the unmeasured eye (a dark closet, indoor ambient lighting, outdoor lighting, and flashlight illumination) to alter pupil size. Linear regression was performed modeling the indexed results N and I against pupil size.

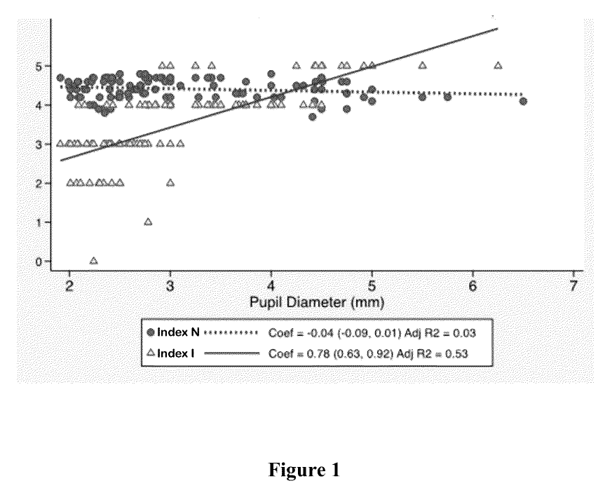

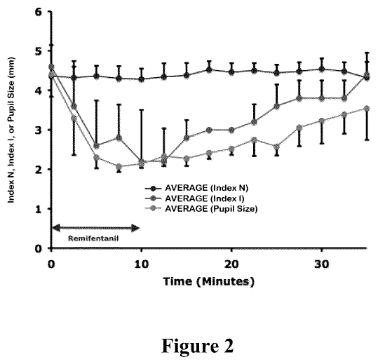

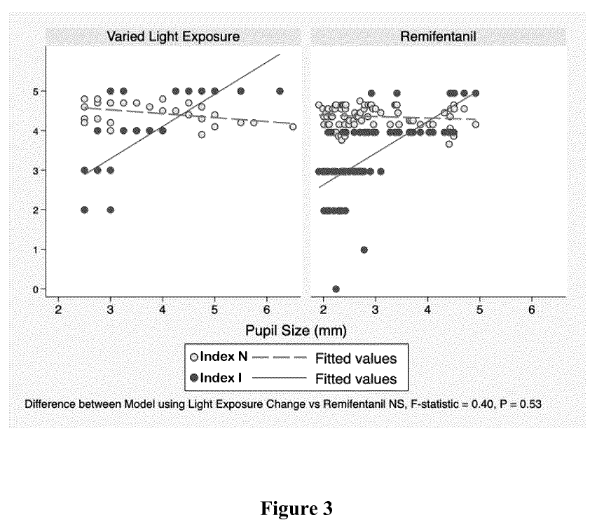

Subjects were 48±10 years old, weighed 73±8 kg; 6 were female. Pupil size decreased progressively with higher remifentanil concentration and greater consensual light stimulation. Pupilometer I recorded a progressive decline in the Index I as pupil size decreased, whereas the pupilometer N readings remained unchanged (FIGS. 1 and 2). A strong relationship between Index I of pupilometer I and pupil size was observed (coefficient=0.78 [0.63, 0.92] aR2=0.53), whereas no significant relationship between Index N and pupil size was observed (coefficient=−0.04 [−0.09, 0.01], aR2=0.03). Joint hypothesis testing demonstrated a statistically significant difference in slope between the two models (F-statistic=99, P<0.0001). The relationship between the measures of light reflex quality did not differ between the two methods of altering pupil size (FIG. 3).

FIG. 1 shows Index N and Index I values in 10 subjects as pupil size was altered by remifentanil in 5 subjects and by directing light in the opposite eye in 5 subjects. The Y axis represents the value of Index N or Index I. The difference in slope was significant, P<0001 between the Index N and Index I.

FIG. 2 provides data in which a remifentanil infusion was used to constrict the pupil while Index N and Index I measurements were taken every 2.5 minutes. Pupil size was the average of the Index N and Index I values which were nearly identical. Note that as the pupil decreased in size, Index N was unchanged whereas the Index I was depressed in diameters below 3.8 mm.

FIG. 3 shows that the relationship between the measures of the quality of the light reflex (Index I and Index N) and pupil size did not differ between two methods of altering the size of the pupil.

Thus, these results indicated that pupilometer I showed a significant decline in indexed output (Index 1) as pupil size decreased, while pupilometer N (Index N) remained stable across the same range. Linear regression revealed a strong dependence of Index I on pupil size (coefficient=0.78, aR²⁼0.53), while Index N showed no significant correlation (coefficient=−0.04, aR²=0.03).

We conclude that in this sample of healthy volunteers, the two indexed measures indicating pupillary reactivity differed markedly between instruments when pupil size was altered by opioid or consensual light stimulation. The separation of the two responses was noted to be close to pupil sizes below 3.5 mm. This is the same diameter at or below which restriction of iris movement following a light stimulus has been observed. There are patients, especially the elderly, those with diabetes, and others receiving opioid medication, that may have constricted pupils without any abnormal or deteriorating neurologic deterioration. Thus, it is important to have a measure that evaluates the quality of the light reflex across the entire range of pupil diameters.

These findings validate the effectiveness of the invention in providing an output unaffected by baseline pupil constriction and support its clinical utility in evaluating true pupillary responsiveness.

While the foregoing has described what are considered to be the best mode and other examples, it is understood that various modifications may be made therein and that the subject matter disclosed herein may be implemented in various forms and examples, and that they may be applied in numerous applications, only some of which have been described herein. It is intended by the following claims to claim any and all modifications and variations that fall within the true scope of the present concepts.

We claim:

1. A pupillometer comprising:
a camera configured to capture images of a subject's eye;
a light source configured to emit a light stimulus;
a display; and
a microprocessor comprising an algorithm that processes image data captured by
the camera, evaluates the pupillary response to the light stimulus and generates an indexed output representing pupillary light reflex reactivity, wherein the indexed output is substantially invariant to changes in baseline pupil size by administration of an opioid medication or any other medication that can affect the size of the pupil to the subject prior to the light stimulus.

2. The pupillometer of claim 1, wherein the algorithm normalizes the measured pupil response relative to an expected pupil diameter under unstimulated conditions.

3. The pupillometer of claim 1, wherein the algorithm applies a correction factor to compensate for opioid-induced miosis.

4. The pupillometer of claim 1, wherein the indexed output is scaled between 0 and 5, with scores below 3 indicating abnormal or reduced pupillary light reflex.

5. The pupillometer of claim 1, wherein the indexed output remains unchanged when pupil size is altered by ambient light stimulation of the contralateral eye.

6. The pupillometer of claim 1, wherein the algorithm includes a machine learning model trained to distinguish between physiological and pharmacological causes of pupil constriction.

7. The pupilometer of claim 1, wherein the medication is an opioid medication.

8. The pupilometer of claim 1, wherein the medication is propofol.
